(19) United States
(12) Patent Application Publication
(10) Pub. No.: US 2009/0143670 A1
(43) Pub. Date: Jun. 4, 2009

(54) OPTICAL TRACKING CAS SYSTEM

(76) Inventor: Emmanuel DAIGNEAULT, Montreal (CA)

Correspondence Address: OGILVY RENAULT LLP, 1981 MCGILL COLLEGE AVENUE, SUITE 1600, MONTREAL, QC, H3A2Y3, CA

(21) Appl. No.: 12/325,011

(22) Filed: Nov. 28, 2008

Related U.S. Application Data
(60) Provisional application No. 60/991,393, filed on Nov. 30, 2007.

(51) Int. Cl. A61B 5/05

(52) U.S. Cl. 600/424

(57) ABSTRACT

A computer-assisted surgery system for tracking an object during surgery comprises two trackable devices secured to two parts of an object. The devices each have optical elements arranged in geometrical patterns. The devices are secured separately to the object so that the devices are at least partially detectable from an overlapping range of directions, so that a combinative geometrical pattern is defined from a combination of at least part of the optical elements from the trackable devices. A sensor unit detects tracking data on any tracked geometrical pattern. A pattern identifier identifies, from known pattern data for the geometrical patterns, which of the geometrical patterns is being tracked. A position and orientation calculator calculates position and orientation of the object as a function of tracking data on an identified geometrical pattern and of a known spatial relation between the identified geometrical pattern and the object. A method for tracking an object is also provided.

### CROSS-REFERENCE TO RELATED APPLICATION

This patent application claims priority on U.S. Provisional Patent Application No. 60/991,393, filed on Nov. 30, 2007.

### FIELD OF THE APPLICATION

The present specification relates to computer-assisted surgery and, more particularly, to instrumentation used for the tracking of surgical tools or other objects during computer-assisted surgery.

### BACKGROUND OF THE ART

Tracking of surgical instruments or tools is an integral part of Computer-Assisted Surgery (hereinafter CAS). The tools are tracked for position and/or orientation in such a way that information pertaining to body parts is obtained. The information is then used in various interventions with respect to the body, such as bone alterations, implant positioning, incisions and the like.

Two types of tracking systems are commonly used. The active tracking systems provide a transmitter on the tool to be tracked, which transmitter emits signals to be received by a processor of the CAS system, which will calculate the position and/or orientation of the tool as a function of the signals received. The transmitters of the active tracking systems are powered, for instance by being wired to the CAS system or by being provided with an independent power source, so as to emit signals.

Passive tracking systems do not provide active transmitters on the tools, and therefore represent fewer issues pertaining to sterilization. The CAS system associated with passive tracking has an optical sensor apparatus provided to visually detect optical elements on the tools. The optical elements are passive, whereby no power source is associated therewith.

In order to obtain values for position and/or orientation, the optical elements must be in the line of sight of the optical sensor apparatus. Accordingly, with passive tracking systems, surgery takes place in a given orientation as a function of the required visibility between the optical sensor apparatus and the optical elements.

Some passive tracking systems use an optical trackable device connected to the object to be tracked. The tracking of the trackable device allows the calculation of position and orientation data for the object.

However, the optical trackable devices define geometrical patterns of optical elements with a relatively small distance between optical elements. Therefore, this relatively small distance between optical elements increases the tolerance error and reduces accuracy.

### SUMMARY

It is therefore an aim of the present description to provide a tracker device that addresses issues pertaining to the prior art.

Therefore, according to one aspect, there is provided a computer-assisted surgery system for tracking an object during surgery, the system comprising: a first trackable device adapted to be secured to a first part of the object, the first trackable device having a first plurality of optical elements arranged in a first geometrical pattern; a second trackable device adapted to be secured to a second part of the object, the second trackable device having a second plurality of optical elements arranged in a second geometrical pattern, the first and the second trackable device being secured separately to the object in such a way that the first and the second trackable device are at least partially detectable from an overlapping range of directions so that a combinative geometrical pattern is defined from a combination of at least part of the optical elements from the first trackable device and from the second trackable device; a sensor unit for detecting tracking data on any tracked one of the first, the second and the combinative geometrical pattern; a pattern identifier for identifying, from known pattern data for the geometrical patterns, which one of the first, the second and the combinative geometrical pattern is being tracked; and a position and orientation calculator for calculating a position and orientation of the object as a function of tracking data on said identified one of the geometrical patterns and of a known spatial relation between said identified one of the geometrical patterns and the object.

Further, according to another aspect, there is provided a computer-assisted surgery system for tracking an object during surgery, the system comprising: a first trackable device adapted to be secured to a first part of the object, the first trackable device having a first plurality of optical elements arranged in a first geometrical pattern; a second trackable device adapted to be secured to a second part of the object, the second trackable device having a second plurality of optical elements arranged in a second geometrical pattern, the first and the second trackable device being secured separately to the object in such a way that the first and the second trackable device are at least partially detectable from an overlapping range of directions so that a combinative geometrical pattern is defined from a combination of at least part of the optical elements from the first trackable device and from the second trackable device; a sensor unit for detecting tracking data on any tracked one of the first, the second and the combinative geometrical pattern; a pattern identifier for identifying, from known pattern data for the geometrical patterns, which one of the first, the second and the combinative geometrical pattern is being tracked; and a position and orientation calculator for calculating a position and orientation of the object as a function of tracking data on said identified one of the geometrical patterns and of a known spatial relation between said identified one of the geometrical patterns and the object.

### BRIEF DESCRIPTION OF THE DRAWINGS

FIG. 1A is a schematic view of an object featuring a pair of trackable devices each having its own geometrical pattern, in accordance with a first embodiment;

FIG. 1B is a schematic view of the object of FIG. 1, with a geometrical pattern defined with both trackable devices;

FIG. 2 is a perspective view of another trackable device used in accordance with a second embodiment;

FIGS. 3A and 3B are schematic view of two of the trackable device of FIG. 2 defining geometrical patterns in accordance with the second embodiment;

FIG. 4 is a Computer-Assisted Surgery (CAS) system using the trackable devices of FIGS. 1A and 1B and FIG. 2; and

FIG. 5 is a flow chart illustrating of a method for tracking an object during CAS.

### DESCRIPTION OF THE PREFERRED EMBODIMENTS

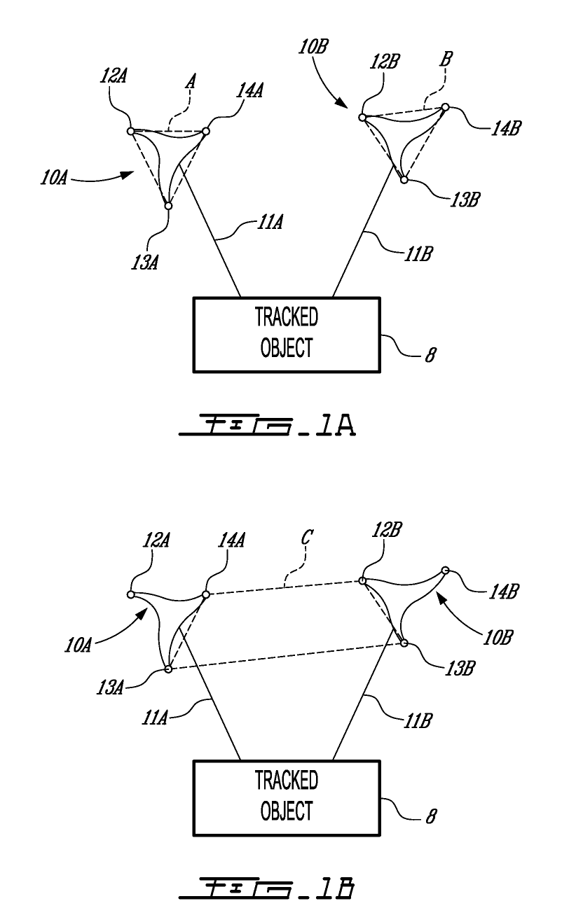

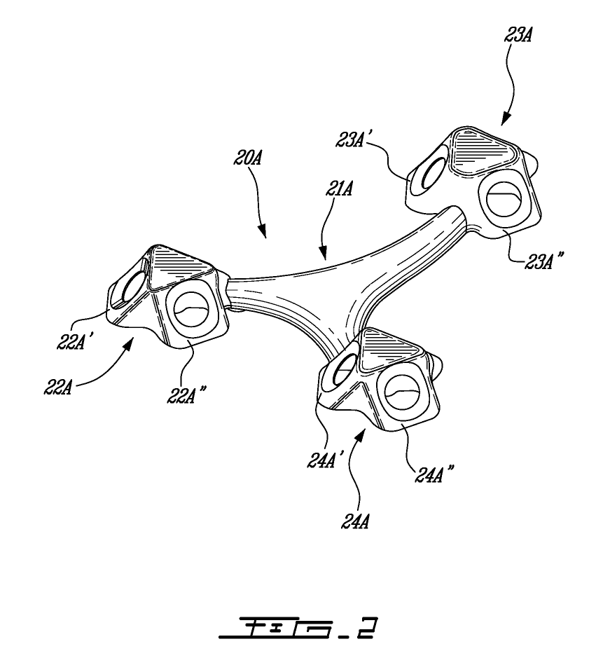

Referring to the drawings and more particularly to FIG. 1A, an object 8 to be tracked (e.g., tracked element) is shown having a pair of trackable devices, namely trackable devices 10A and 10B (also concurrently referred to as trackable device 10).

Each of the trackable devices 10 has a support 11 that interrelates tracker members 12, 13 and 14 to the tracked object 8 (e.g., instruments and surgical tools used in CAS, bone element, axes or frames of reference associated with the bone element, C-arm for fluoroscopy). Although not described in detail hereinafter, the support 11 is anchored to the tracked object by various mechanical means so as to be fixed to the tracked object 8.

In order for an object to be tracked in space for position and orientation, at least two points associated with the object should be known. With two points, the object 8 can be tracked for position and orientation under specific conditions (e.g., object and the two tracked points being collinear, and no view interruption after calibration). A geometrical pattern of three nonlinear trackable points is commonly used for six-degree-of-freedom tracking, and more trackable points can be used for increased precision in the tracking.

The support 11 supports the tracker members 12, 13 and 14 in a given geometry, such that an optical sensor apparatus of a CAS system visually recognizes the given geometry. With the tracking of the patterns of the tracker device 10, the CAS system calculates a position and/or orientation of the tracked object associated with the tracker devices 10.

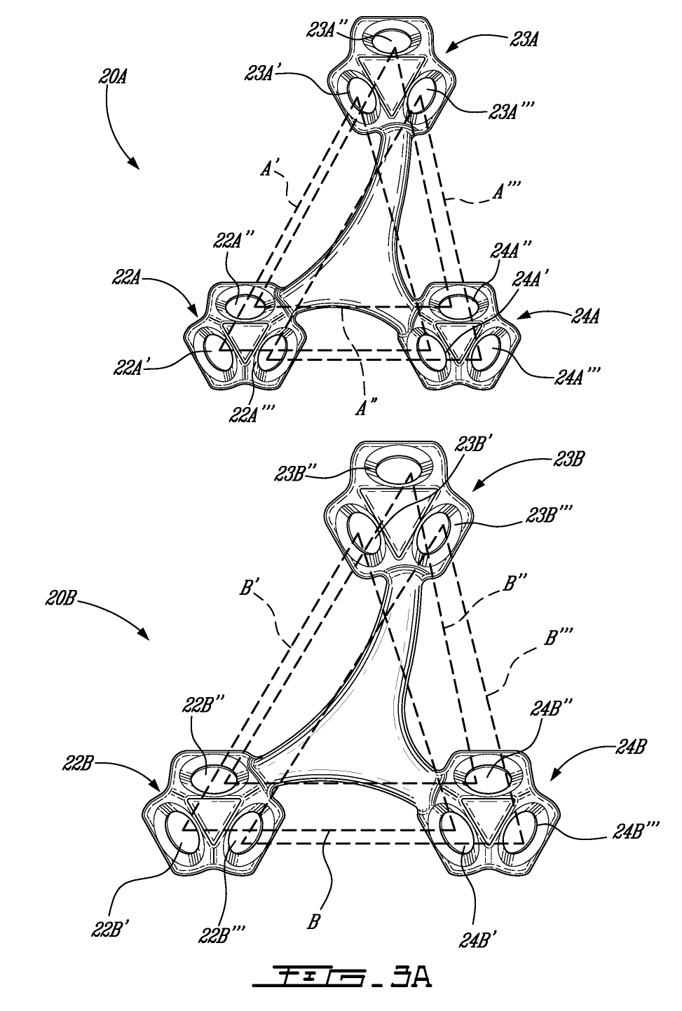

The tracker members 12-14 are optical elements that constitute the geometrical patterns and are thus visually detectable by the optical sensor apparatus of the CAS system. In an embodiment, the tracker members 12-14 are retro-reflective spheres, but other shapes and types of tracker members can be used, as described, for instance, below for FIG. 2.

Referring to FIG. 1A, the tracker members 12A-14A of the trackable device 10A form a triangular geometrical pattern A, whereas the tracker members 12B-14B of the trackable device 10B form a triangular geometrical pattern B. From a plan view, the triangular geometrical pattern A and B are both scalene triangles, with the geometrical pattern A and B representing different geometries from the plan view.

Therefore, the CAS system calculates the position and orientation of the tracked object 8 from the optical tracking of either one of the triangular geometrical patterns A and B. As the tracking is optical, there should be a line of sight between the optical sensor apparatus and the trackable devices 10A or 10B. It is therefore advantageous to have two trackable devices 10A and 10B, to increase the range of visibility of tracked object 8.

Referring to FIG. 1B, it is seen that another detectable geometrical pattern C is formed from tracker members of both trackable devices 10A and 10B. The geometrical pattern C is a quadrilateral formed by the tracker members 13A and 14B from the trackable device 10A, and the tracker members 12B and 13B from the trackable device 10B. Accordingly, the optical sensor apparatus of the CAS (described hereinafter) recognizes and tracks any one of the three patterns A, B (FIG. 1A) or C (FIG. 1B), for the CAS to calculate the position and orientation of the tracked object 8.

In defining a third or combinative geometrical pattern C, the CAS ensures that the third pattern C is different than the other two patterns A, B from a plan view (with the other two patterns being different from one another, as mentioned previously). In the embodiment illustrated in FIGS. 1A and 1B, the geometrical patterns are two different scalene triangles (A and B) and a quadrilateral (C).

The third geometrical pattern C advantageously has a greater distance between its optical elements. Accordingly, the greater distance reduces the error in tracking objects 8. It is also considered to track pentagonal, hexagonal, and other polygonal geometrical patterns.

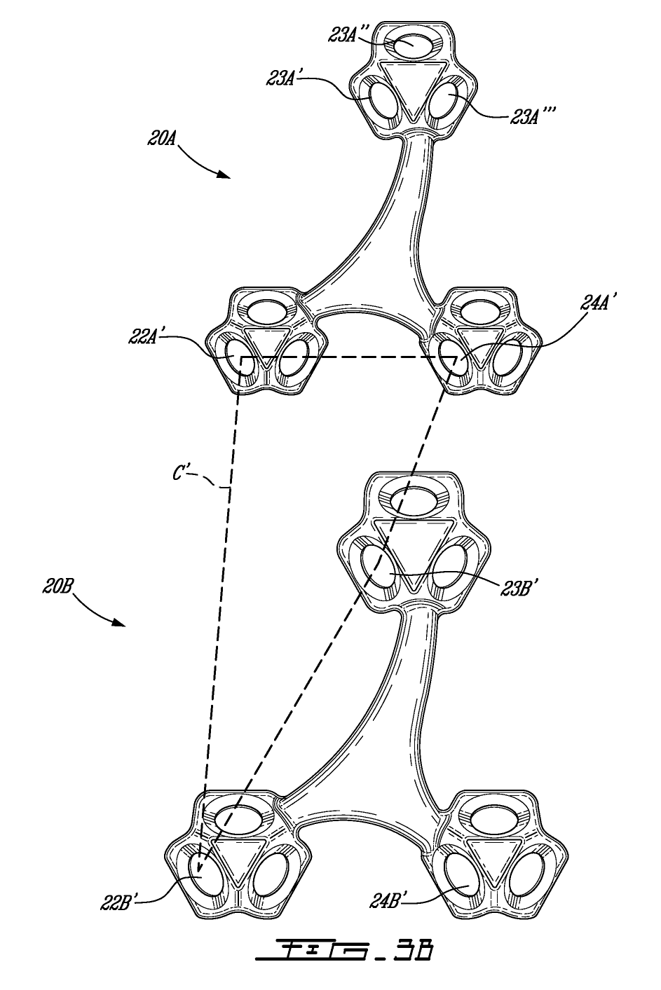

Referring to FIGS. 2, 3A and 3B, an alternative to retroreflective spheres is described. In FIGS. 2, 3A and 3B, the patterns A, B and C are obtained from multifaceted tracker devices 20A and 20B, and the object or tracked object 8 is not shown for clarity purposes. Reference is made to United States Patent Publication No. 2007/0100325, published on May 3, 2007, by Jutras et al., in which such multifaceted tracker devices 20 are described.

In FIG. 3A, pattern A′ is defined by optical elements 22A′, 23A′, and 24A′ of the trackable device 20A. Pattern B′ is defined by optical elements 22B′, 23B′, and 24B′ of the trackable device 20B. In FIG. 3B, pattern C′ is defined by optical elements 22A′, 24A′, 22B′ and 23B′ of the trackable devices 20A and 20B. Any other combination is considered, using other optical elements (e.g., 23A″, 23A′″).

In the embodiment of FIGS. 2, 3A and 3B, each tracker device 20 has three sets of three detectable elements. The detectable elements are retroreflective surfaces of circular shape, although other shapes are also considered. The retroreflective surfaces are made of a retroreflective material that are detectable by the optical sensor apparatus associated with the CAS system. For instance, the material Scotch-Lite™ is suited to be used as a retroreflective surface.

As the optical elements must be in a given geometrical pattern to be recognized by the optical sensor apparatus of the CAS system, the optical elements are regrouped into one embodiment in sets of three.

The sets of optical elements are strategically positioned with respect to one another so as to optimize a range of visibility of the tracker devices 20A and 20B. More specifically, the sets are positioned such that once the optical sensor apparatus of the CAS system loses sight of one of the sets, another set is visible. This ensures the continuous tracking of the trackable devices 20A and 20B within a given range of field of view.

The sets each form a geometrical pattern that is recognized by the optical sensor apparatus of the CAS system. The combination of circular openings and retro-reflective surface gives a circular shape to the optical elements. Depending on the angle of view of the optical sensor apparatus, these circles will not always appear as being circular in shape. Therefore, the position of the center of the circles can be calculated as a function of the shape perceived from the angle of view by the optical sensor apparatus.

It is preferred that the three triangles of the three different sets of optical elements be of different shape, with each triangle being associated with a specific orientation with respect to the tool. Alternatively, the three triangles formed by the three different sets may be the same, but the perceived shape of the circular reflective surfaces should then be used to identify which of the three sets of reflective surfaces is seen.

Although triangular geometrical patterns are illustrated, it is contemplated to use other geometrical patterns, such as lines and various polygonal shapes.

It is pointed out that a calibration of the object with the trackable devices 20 thereon is preferably performed prior to the use of the trackable devices 20, to calibrate a position and/or orientation of each of the detectable geometrical patterns (i.e., A, B and C, amongst others) with respect to the object.

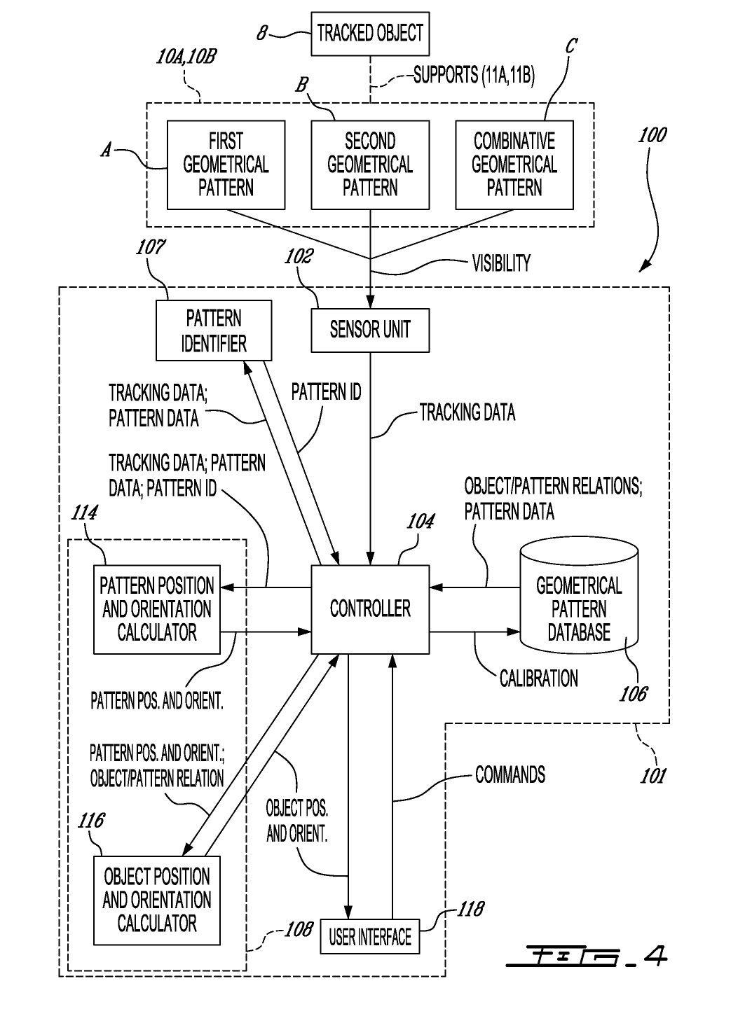

Referring to FIG. 4, an optical tracking computer-assisted surgery system using the tracker devices 10A and 10B is generally illustrated at 100. The computer-assisted surgery system 100 incorporates the tracker devices 10A and 10B, as secured to a tracked object 8 using supports 11A and 11B, as described above. It is however noted that the trackable devices 20A and 20B or other embodiments of trackable devices may also be used.

In accordance with FIGS. 1A and 1B, the tracker devices 10A and 10B each provide at least one detectable geometrical pattern (A and B, respectively in FIG. 1A), and concurrently provide at least another different geometrical pattern (C in FIG. 1B). The recognition of the at least three geometrical patterns may result from a calibration performed in the first steps of use of the computer-assisted surgery system.

The computer-assisted surgery system 100 has a tracking system 101, which is typically a computer having a processor. The tracking system 101 has a sensor unit 102 (i.e., an optical sensor apparatus) provided in order to visually track the tracker members 12-14 of the trackable devices 10A and 10B. Typically, the sensor unit 102 has a 3D camera which involves a pair of sensors (e.g., a Navitrack™ by ORTHOsoft Inc.). The sensor unit 102 also has an image processing unit (not shown) that analyses the acquired images in order to identify optical elements on the images and produce tracking data regarding their coordinates. It is noted that the images acquired by the sensor unit 102 may not include all the optical elements of the first trackable device and the second trackable device and that, accordingly, not all three geometrical patterns A, B, C will be detected at a same time.

A controller 104 is connected to the sensor unit 102. Therefore, the controller 104 receives the tracking data from the sensor unit 102.

A database 106 is provided so as to store the geometrical pattern data. More specifically, the various patterns of the tracker devices 10A and 10B are stored in the database 106. Similarly, the spatial relation between the tracked object and the patterns is stored in the database 106. The tracked object/pattern spatial relation may result from a calibration performed in the first steps of use of the computer-assisted surgery system.

A pattern identifier 107 is associated with the controller 104. The pattern identifier 107 receives the tracking data from the sensor unit 102 and the geometrical pattern data from the database 106, so as to identify which one of the patterns of the tracker devices 10A and/or 10B is being tracked. If multiple patterns are visible, it is preferred that the pattern having the greatest distance between its optical elements (e.g., pattern C in FIG. 1B) or the most points (e.g., quadrilateral over triangles) be selected to reduce the error.

The position and orientation calculator 108 is associated with the controller 104. The position and orientation calculator 108 calculates the position and orientation of the object. The position and orientation calculator 108 comprises a pattern position and orientation calculator 114 and a tracked object position and orientation calculator 116.

The pattern position and orientation calculator 114 receives the tracking data and the identification of the tracked pattern from the controller 104 so as to calculate the position and orientation of the tracked pattern in space.

The tracked object position and orientation calculator 116 receives the position and orientation of the tracked pattern from the controller 104, as well as the spatial relation between the tracked pattern and the tracked object, which is stored in the database 106. It then combines this information so as to calculate the position and orientation of the tracked object.

The position and orientation of the tracked object is sent to the user interface 118, such that the user of the computer-assisted surgery system obtains information pertaining to the position and orientation of the tracked object in the various forms known to computer-assisted surgery (e.g., visual representation, numerical values such as angles, distances, etc.). It is pointed out that the database 106 may as well be part of the controller 104, the pattern identifier 107 or the position and orientation calculator 108.

It is noted that the computer-assisted surgery system 100 may include other modules to perform other functions typical to computer-assisted surgery, such as calculations of surgical parameters, presentation of visual data, etc. For simplicity purposes, the present disclosure is limited to the tracking of trackable references to provide position and orientation data for objects such as bones and surgical tools.

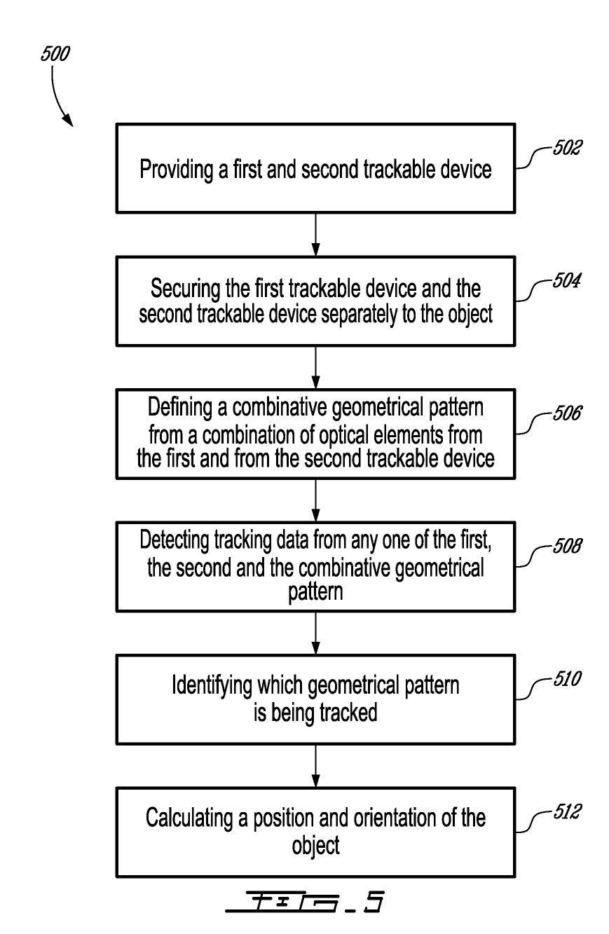

FIG. 5 shows a method 500 for tracking an object during computer-assisted surgery. For example, the method 500 may be implemented using the tracking system 100 of FIG. 4. In step 502, there are provided a first and a second trackable device such as the trackable devices 10A, 10B of FIGS. 1A and 1B or the trackable devices 20A, 20B of FIGS. 2, 3A and 3B.

In step 504, the trackable devices are secured separately to a first and a second part of the object to be tracked, in such a way that the optical elements from the first and the second trackable devices are detectable from an overlapping range of directions. From some directions, images acquired by the sensor unit include only the optical elements of the first trackable device. From some other directions, images include only the optical elements of the second trackable device. From some yet other directions, the images overlap between optical elements of the first trackable device and of the second trackable device, thereby overlapping. More specifically, according to an embodiment, some of the optical elements from the first trackable device and some of the optical elements of the second trackable device are visible from a given range of directions.

Accordingly, in step 506, a combinative geometrical pattern (e.g. pattern C of FIG. 1B or pattern C′, C″ or C′″ of FIG. 3B) is defined from a combination of the optical elements from the first and the second trackable devices visible from the given range of direction.

In step 508, tracking data is detected on any tracked one of the first, the second and the combinative geometrical pattern. The tracking data is obtained, for example, using the sensing device 102 of FIG. 4.

In step 510, the geometrical pattern to be tracked is identified from known pattern data on the spatial configurations of the geometrical patterns. This is performed, for example, using the pattern identifier 107 of FIG. 4. It is pointed out that the system is configured to continuously track the object. Accordingly, the pattern identifier 107 may switch the tracking from one of the patterns to another, in accordance with the pattern that is visible to the sensor unit 102. If more than one geometrical pattern is detected by the pattern identifier 107, the combinative geometrical pattern may be prioritized as it is greater in dimension than the first and the second geometrical patterns.

In step 512, the position and orientation of the tracked object is calculated from the tracking data on the identified one of the geometrical patterns and a known spatial relation between the identified one of the geometrical patterns and the tracked object. This is calculated using, for example, the position and orientation calculator 108 of FIG. 4.

While illustrated in the block diagram as groups of discrete components communicating with each other via distinct data signal connections, it will be understood by those skilled in the art that the illustrated embodiments may be provided by a combination of hardware and software components, with some components being implemented by a given function or operation of a hardware or software system, and many of the data paths illustrated being implemented by data communication within a computer application or operating system. The structure illustrated is thus provided for efficiency of teaching the described embodiment.

The embodiments described above are intended to be exemplary only. The scope of the invention is therefore intended to be limited solely by the appended claims.

1. A computer-assisted surgery system for tracking an object during surgery, the system comprising:
a first trackable device adapted to be secured to a first part of the object, the first trackable device having a first plurality of optical elements arranged in a first geometrical pattern;
a second trackable device adapted to be secured to a second part of the object, the second trackable device having a second plurality of optical elements arranged in a second geometrical pattern, the first and the second trackable device being secured separately to the object in such a way that the first and the second trackable device are at least partially detectable from an overlapping range of directions so that a combinative geometrical pattern is defined from a combination of at least part of the optical elements from the first trackable device and from the second trackable device;
a sensor unit for detecting tracking data on any tracked one of the first, the second and the combinative geometrical pattern;
a pattern identifier for identifying, from known pattern data for the geometrical patterns, which one of the first, the second and the combinative geometrical pattern is being tracked; and
a position and orientation calculator for calculating a position and orientation of the object as a function of tracking data on said identified one of the geometrical patterns and of a known spatial relation between said identified one of the geometrical patterns and the object.

2. The computer-assisted surgery system as claimed in claim 1, wherein said first trackable device has a secondary first plurality of optical elements arranged in a secondary first geometrical pattern and said second trackable device has a secondary second plurality of optical elements arranged in a secondary second geometrical pattern, in such a way that the secondary first and the secondary second plurality of optical elements are at least partially detectable from another overlapping range of directions, so that a secondary combinative geometrical pattern is defined from a combination of at least part of the optical elements from said secondary first and from said secondary second plurality of optical elements.

3. The computer-assisted surgery system as claimed in claim 1,
wherein said first plurality of optical elements comprise three optical elements defining said first geometrical pattern in a first triangular pattern; and
wherein said second plurality of optical elements comprise three optical elements defining said second geometrical pattern in a second triangular pattern.

4. The computer-assisted surgery system as claimed in claim 1, wherein the pattern identifier prioritizes the combinative geometrical pattern when the combinative geometrical pattern and at least one of the first and the second geometrical patterns are identified concurrently.

5. The computer-assisted surgery system as claimed in claim 1, wherein the pattern identifier receives the known pattern data pertaining to said first and second geometrical pattern from a database.

6. The computer-assisted surgery system as claimed in claim 5, wherein the pattern identifier receives the known pattern data pertaining the combinative geometrical pattern from a calibration performed in situ.

7. The computer-assisted surgery system as claimed in claim 1, wherein said first trackable device and said second trackable device are secured to a surgical instrument for tracking said surgical instrument during computer-assisted surgery.

8. The computer-assisted surgery system as claimed in claim 1, wherein said first trackable device and said second trackable device are secured to a bone element for tracking said bone during computer-assisted surgery.

9. The computer-assisted surgery system as claimed in claim 1, wherein the combinative geometrical pattern has more than three of said optical elements.

10. A method for tracking an object during computer-assisted surgery, the method comprising:
providing a first trackable device having a first plurality of optical elements arranged in a first geometrical pattern, and a second trackable device having a second plurality of optical elements arranged in a second geometrical pattern;
securing the first trackable device and the second trackable device separately to a first part and a second part of the object, in such a way that at least some optical elements from the first and the second trackable device are detectable from a given range of directions;
defining a combinative geometrical pattern from a combination of the optical elements from the first and from the second trackable device visible from the given range of directions;
detecting tracking data on any tracked one of the first, the second and the combinative geometrical pattern;
identifying, from known pattern data for the geometrical patterns, which one of the first, the second and the combinative geometrical pattern is being tracked; and
calculating a position and orientation of the object from said tracking data on said identified one of the geometrical patterns and a known spatial relation between said identified one of the geometrical patterns and the object.

11. The method according to claim 10, wherein identifying the geometrical pattern being tracked comprises prioritizing an identification of the combinative geometrical pattern over any one of the first and the second geometrical pattern when at least two of the geometrical patterns are detected concurrently.

12. The method according to claim 10, wherein defining the combinative geometrical pattern comprises defining the combinative geometrical pattern from more than three optical elements.

13. The method according to claim 10, wherein securing the first trackable device and the second trackable device comprises securing the first trackable device and the second trackable device to a surgical instrument.

14. The method according to claim 10, wherein securing the first trackable device and the second trackable device comprises securing the first trackable device and the second trackable device to a bone.

15. The method according to claim 14, wherein securing the first trackable device and the second trackable device to the bone comprises securing the first trackable device and the second trackable device to a bone model or to a cadaver.
